(19) United States
(12) Patent Application Publication
(10) Pub. No.: US 2011/0118594 A1
(43) Pub. Date: May 19, 2011

(54) MEDICAL IMPLANT TARGETING SYSTEM AND METHOD

(76) Inventors: Varad N. Srivastava, Rockford, IL (US); James J. Foskett, Rockford, IL (US); Michael G. Schneider, Rockford, IL (US); Dan Pickerill, Rockford, IL (US); Abtin Spantman, Loves Park, IL (US)

(21) Appl. No.: 13/054,634

(22) PCT Filed: Aug. 19, 2009
(86) PCT No.: PCT/US2009/004717

§ 371 Date: Jan. 18, 2011

Related U.S. Application Data
(60) Provisional application No. 61/190,143, filed on Aug. 26, 2008.

(51) Int. Cl. A61B 5/05

(52) U.S. Cl. 600/424

(57) ABSTRACT

A system (10) and method for targeting a geometric feature (12) in a medical implant (14) located in a patient (16), such as a transverse bore (12) in an intramedullary nail (14) implanted in a femur (18) of a patient (16). The system (10) includes one or more emitters (20), one or more receptors (22), and a processor (24). The emitter(s) emits at least one of an acoustic field, a magnetic field, an electric field, or an electromagnetic field that will be altered by the geometric feature (12), and the receptor(s) (22) detects the altered field and generates a signal (26) responsive to the altered field. The processor receives the signal and generates a representation of the altered field.

### CROSS-REFERENCE TO RELATED APPLICATIONS

This patent application claims the benefit of U.S. Provisional Patent Application 61/190,143, filed on Aug. 26, 2008, the entire disclosure of which is hereby incorporated by reference.

### BACKGROUND

One challenge presented by modern medical techniques is the location of geometric features, such as apertures or bores in medical implants or other objects that have been inserted into a patient. A particularly difficult challenge is presented by intramedullary nails or rods used in orthopedic surgery to help repair bone fractures. It is common for such intramedullary nails to have predrilled bores adjacent both ends of the nail to receive anchors or fasteners that extend through holes drilled in the bone in alignment with the bores of the intramedullary nail. Because the predrilled bores are not visible once the intramedullary nail is inserted into the bone, they must somehow be located and targeted so that the holes in the bone can be drilled in accurate alignment with the predrilled bores using a surgical drill. In this regard, the predrilled bore at the distal end of the location of intramedullary nail is particularly difficult to accurately locate because deformation of the intramedullary nail is common during insertion, with lateral “wandering” of the distal end often being significant.

The most common method for targeting the predrilled bores to assist in aligning the surgical drill is for a surgeon to use x-rays and fluoroscopes to locate the bores, which exposes operating room personnel and patients to increased radiation dosage. Other approaches have been attempted wherein, via various means, an electric or magnetic field is generated from a location in the intramedullary nail, such as by: placing one or more permanent magnets in the nail, temporarily magnetizing the nail, or inserting a field generating probe into the nail. While at least some of these approaches may be suitable for their intended purpose, all of them involve modification of the intramedullary nail, insertion of additional foreign objects into a patient and/or additional invasive procedures. Accordingly, there is a continuing need for improvement in such technology.

### SUMMARY

In accordance with one feature of the claimed subject matter, a system is provided to target a geometric feature in a medical implant located in a patient. The system includes at least one emitter to emit at least one of an acoustic field, a magnetic field, an electric field and an electromagnetic field that will be altered by the geometric feature; at least one receptor to generate a signal responsive to the altered field; and a processor to receive the signal and generate a representation of the altered field.

In one feature, the at least one emitter and the at least one receptor are external to the patient.

As one feature, the system further includes a frame and a carrier mounted to the frame for translation and rotation along and about two axes relative to the medical implant. The at least one emitter and at least one receptor are mounted on the carrier for movement therewith along and about the two axes.

In one feature, the carrier is further mounted to the frame for translation and rotation along and about another axis relative to the medical implant.

According to one feature, the at least one emitter and the at least one receptor are located on the carrier so that the medical implant can be positioned between the at least one receptor and the at least one emitter with the medical implant located in a patient.

As one feature, the at least one emitter and the at least one receptor are located on the carrier so that the medical implant can be positioned to one side of the at least one receptor and the at least one emitter with the medical implant located in a patient.

According to one feature, the system further includes a comparator to compare at least a portion of the representation of the altered field to a shape of the known geometric feature.

In one feature, the system further includes a controller to position the carrier relative to the medical implant in response to the representation of the altered field.

As one feature, the controller includes a comparator to compare at least a portion of the representation of the altered field to a shape of the known geometric feature, and the controller is responsive to the comparator to position the carrier relative to the implant.

According to one feature, the controller is responsive to a representation of relative changes in an intensity of the altered field with respect to a reference coordinate system.

In one feature, the system further includes a surgical tool guide to be targeted at the geometric feature in response to the representation of the altered field.

As one feature, the system further includes a surgical tool guide mounted on the carrier to be targeted at the geometric feature in response to the representation of the altered field.

In one feature, the system further includes a user interface to transmit the representation of the altered field to a user.

According to one feature, the user interface includes at least one of a visual display and an audio signal.

As one feature, the at least one emitter includes a constant DC field emitter.

In one feature, the at least one emitter includes a pulsed DC field emitter.

According to one feature, the at least one emitter includes an AC magnetic field emitter.

As one feature, the at least one emitter includes an AC electric field emitter.

According to one feature, the at least one emitter includes an acoustic emitter.

As one feature, the at least one emitter includes an optical emitter.

In accordance with one feature of the claimed subject matter, a method is provided to target a geometric feature in a medical implant located in a patient. The method includes the steps of:

emitting, at a location external to the implant, at least one of an acoustic field, a magnetic field, an electric field and an electromagnetic field that will be altered by the geometric feature;

detecting the altered field at a location external to the implant; and

generating a representation of the altered field in response to the detecting step.

In one feature, the emitting and detecting steps occur at locations external to the patient.

According to one feature, the method further includes the step of altering the locations of the emitting and detecting steps relative to the medical implant in response to the representation of the altered field.

In one feature, the altering step includes translating the locations along at least one axis relative to the medical implant.

As one feature, the altering step includes rotating the locations about at least one axis relative to the medical implant.

According to one feature, the emitting step includes the step of varying the at least one of an acoustic field, a magnetic field, an electric field and an electromagnetic field.

In one feature, the varying step includes varying a frequency of the at least one of an acoustic field, a magnetic field, an electric field and an electromagnetic field.

As one feature, the varying step includes pulsing the at least one of an acoustic field, a magnetic field, an electric field and an electromagnetic field.

In one feature, the method further includes the step of comparing at least a portion of the representation of the altered field to a shape of the known geometric feature.

In one feature, the method further includes the step of comparing relative changes in intensity of the representation of the altered field over a reference coordinate system.

As one feature, the method further includes the step of altering the locations of the emitting and detecting steps relative to the medical implant in response to the comparing step.

According to one feature, the altering step includes translating the locations along at least one axis relative to the medical implant.

As one feature, the altering step includes rotating the locations about at least one axis relative to the medical implant.

In one feature, the method further includes the step of comparing relative changes in intensity of the representation of the altered field over a reference coordinate system.

As one feature, the method further includes the step of targeting a surgical tool guide relative to the geometric feature in response to the generating step.

According to one feature, the method further includes the step of transmitting the representation of the altered field to a user.

As one feature, the transmitting step includes transmitting the representation of the field to at least one of a visual display and an audio signal.

In accordance with one feature of the claimed subject matter, a system is provided to target a geometric feature in a medical implant located in a patient. The system includes at least one emitter to emit at least one of an acoustic field, a magnetic field, an electric field and an electromagnetic field that will be altered by the geometric feature; at least one receptor to generate a signal responsive to the altered field; and a carrier which houses the at least one of an emitter and at least one of a receptor.

As one feature, the system further includes a processor located in the carrier to receive the signal and generate a representation of the altered field.

According to one feature of the claimed subject matter, the system further includes a processor located external to the carrier to receive the signal and generate a representation of the altered field.

In accordance with one feature of the claimed subject matter, the system further comprises a comparator located in the carrier, to compare at least a portion of the representation of the altered field to a shape of a known geometric feature.

In accordance with one feature of the claimed subject matter, the system further comprises a comparator located external to the carrier, to compare at least a portion of the representation of the altered field to a shape of a known geometric feature.

According to one feature of the claimed subject matter the altered field comprises information of at least one of intensity, frequency, shape and phase of the altered field.

As one feature of the claimed subject matter, the system further comprises a tool guide mounted on the carrier to be targeted relative to the geometric feature in response to the representation of the altered field.

According to another feature of the claimed subject matter, the system further comprises a user interface housed in the carrier to transmit the representation of the altered field to the user.

As another feature of the claimed subject matter, the system further comprises a user interface external to the carrier to transmit the representation of altered field to the user.

In accordance with another feature of the claimed subject matter, the user interface comprises at least one of a visual and an audio signal.

As another feature of the claimed subject matter, at least a portion of the system is disposable.

According to one feature of the claimed subject matter, the system is hand held.

Other objects, features, and advantages of the claimed subject matter will become apparent from a review of the entire specification, including the appended claims and drawings.

### BRIEF DESCRIPTION OF THE DRAWINGS

FIG. 1 is a diagrammatic representation of a one embodiment of the claimed subject matter for targeting a known geometric feature in a medical implant located in a patient; and

FIG. 2 is a somewhat diagrammatic representation showing a structural form for some of the components of the system of FIG. 1.

FIG. 3 is a diagrammatic representation of an embodiment of the claimed subject matter wherein the system embodying the claimed subject matter may be housed in the carrier.

FIG. 4 is a diagrammatic representation of an embodiment of the claimed subject matter wherein the at least some elements of the system embodying the claimed subject matter may be external to the carrier.

FIG. 5 is a somewhat diagrammatic representation showing a structural form for some of the components of the system in FIG. 4.

FIG. 6 is a somewhat diagrammatic representation showing a structural form for some of the components of the system in FIG. 3.

### DETAILED DESCRIPTION

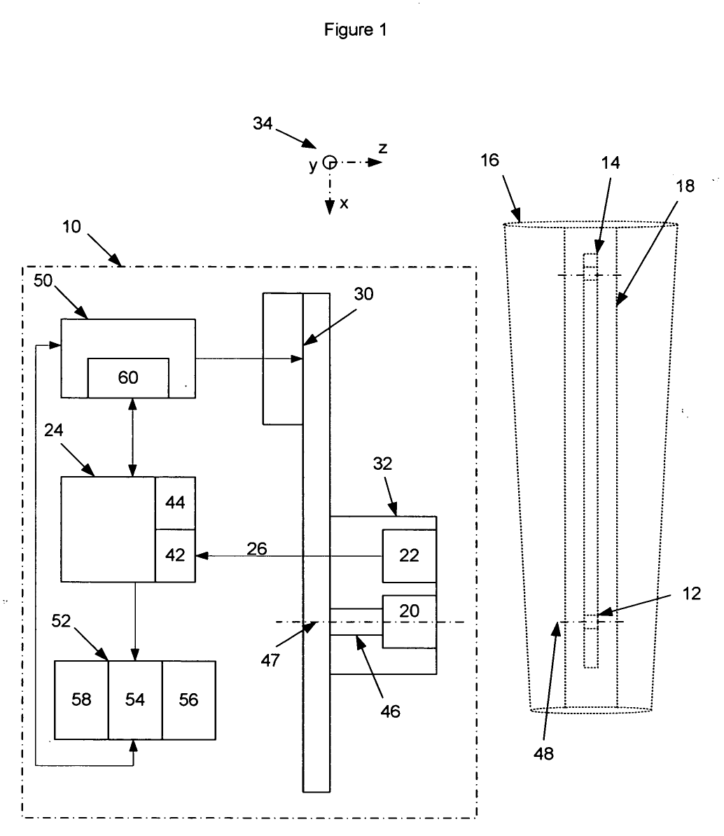

In one embodiment of the claimed subject matter, the system is configured to be secured relative to the patient during the operation of the system. With reference to FIG. 1, a system 10 is shown for targeting a known geometric feature(s) 12, shown in the form of a transverse bore, in a medical implant 14, shown in the form of an intramedullary nail or rod, located in a patient 16, such as in a femur (thigh bone) 18 of a patient 16. The system 10 includes one or more emitters 20, one or more receptors 22, and a processor 24. The emitter(s) 20 emits at least one of an acoustic field, a magnetic field, an electric field, or an electromagnetic field that will be altered by the geometric feature 12, and the receptor(s) 22 detects the altered field and generates a signal 26 responsive to the altered field. The processor 24 receives the signal 26 and generates a representation of the altered field. In this regard, testing has shown that a known geometric feature 12 in a surgical implant 14 will produce a detectable altered field in a predictable manner that will allow the location and orientation of the geometric feature 12 to be accurately determined based on the representation of the altered field.

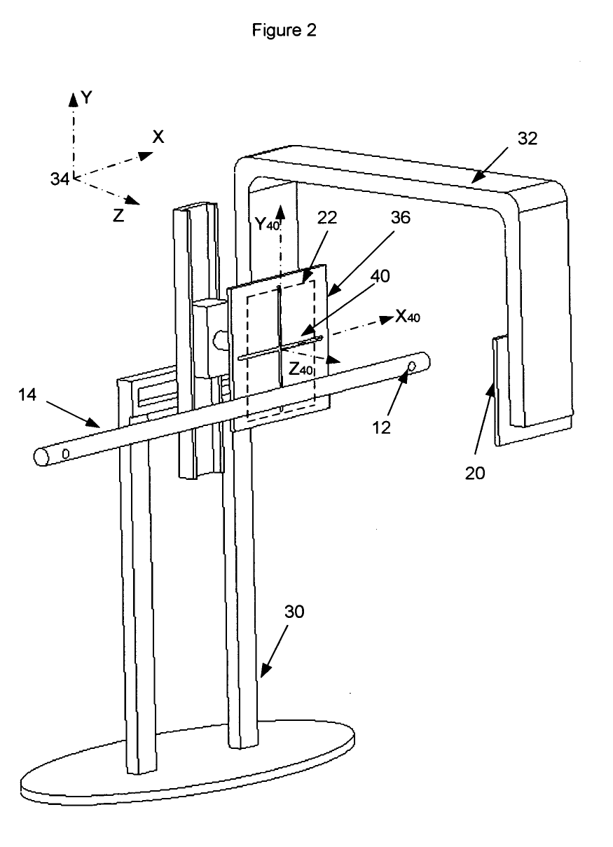

With reference to both FIGS. 1 and 2 which show embodiments of the claimed subject matter configured to be secured relative to the patient during the operation of the device; the system 10 includes a structural frame 30 and a carrier 32 mounted to the frame 30 for translation and rotation along and about two axes relative to the medical implant 14 (for purposes of illustration, the patient 16 and femur 18 are not shown in FIG. 2). The frame 30 can be supported relative to a patient in any suitable fashion, including for example, being anchored to an operating room floor, attached to an operating table, suspended from a ceiling, cantilevered from a wall, or supported on an independent and/or repositionable base of the frame 30. Preferably, the two axes are X and Y axes of a Cartesian coordinate system 34 that is fixed relative to the frame 30, as well as the patient 16 and the medical implant 14, both of which preferably maintain a constant position with respect to the frame 30 during operation of the system 10. As one option, in addition to the X and Y axes, the carrier 32 can be further mounted to the frame 30 for translation and rotation along and about the Z axis of the coordinate system 34 relative to the medical implant 14. The emitter(s) 20 and the receptor(s) 22 are mounted on the carrier 32 for movement therewith along and about the X, Y and Z axes relative to the patient 16 and the implant 14. In this regard, the receptor(s) 22 are shown in FIG. 2 in connection with a receptor 36 that lays in a plane normal to and preferably centered on a reception/emission direction vector defined by the receptor(s) 22 and emitter(s) 20.

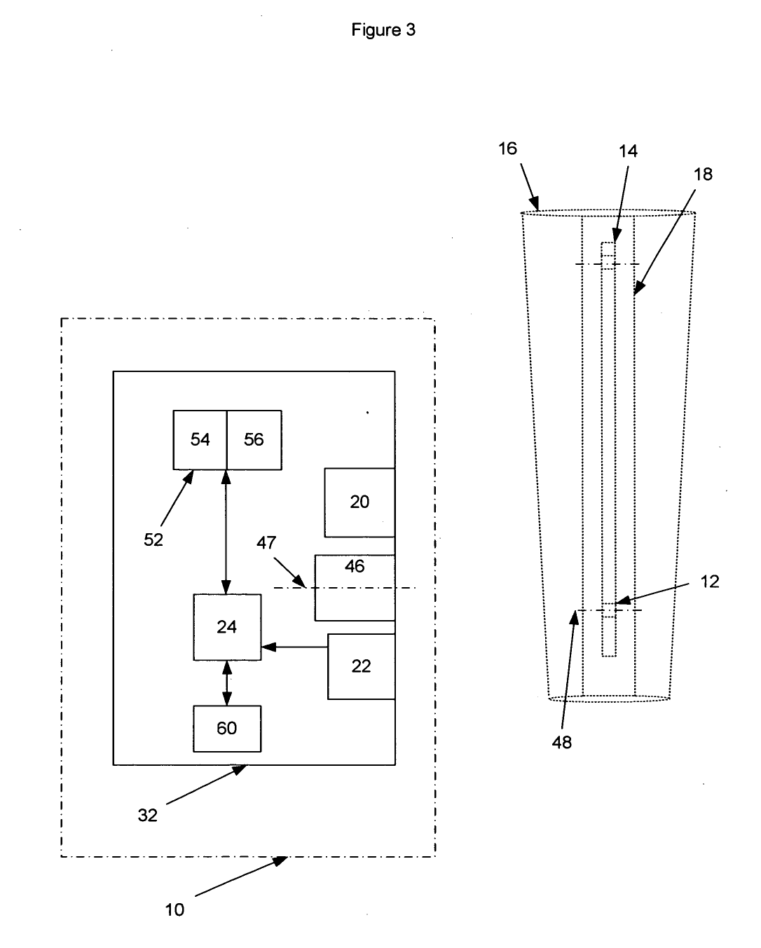

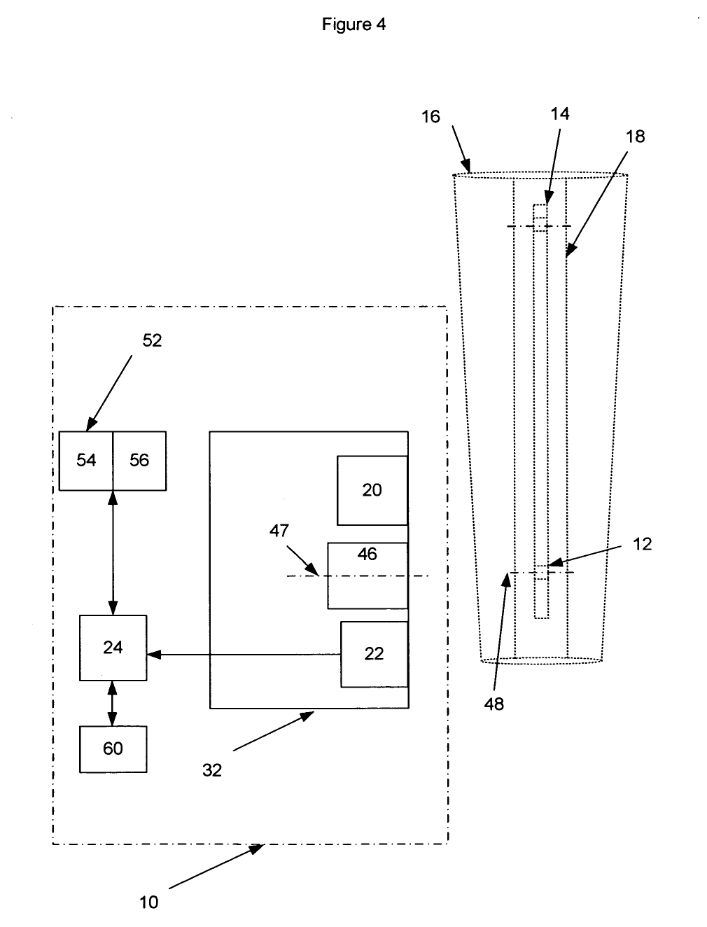

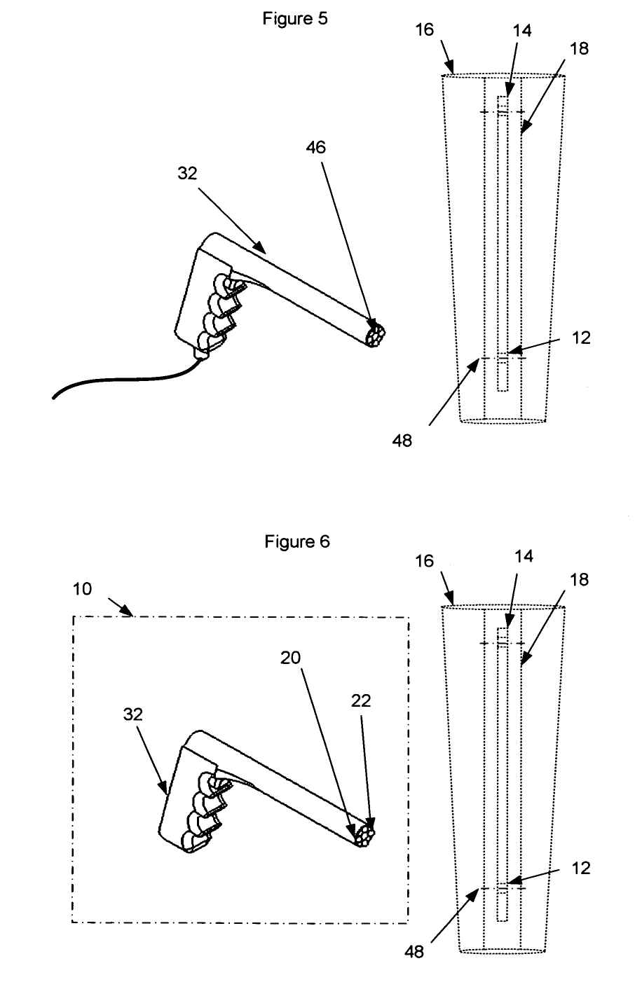

In the embodiment of FIG. 2 which shows an embodiment of the claimed subject matter configured to be secured relative to the patient during the operation of the device; the emitter(s) 20 and receptor(s) 22 are located on the carrier 32 so that the medical implant 14 can be positioned between the emitter(s) 20 and receptor(s) 22 with the medical implant 14 located in a patient. This allows for a through measurement wherein the receptor(s) 22 detect the altered field after it has passed through the patient 16 (through measurement). On the other hand, in the embodiments of FIG. 1, FIG. 3, FIG. 4, FIG. 5 and FIG. 6 the emitter(s) 20 and the receptor(s) 22 are located on the carrier 32 so that the medical implant 14 can be positioned to one side of the emitter(s) 20 and receptor(s) 22 with the implant 14 located in the patient 16. This allows for a reflective measurement wherein the receptor(s) 22 detect the altered field after it has been reflected back from the implant 14 and patient 16.

The processor 24 may generate the representation of the altered field in three dimensions, with two of the dimensions representing the spatial dimensions of the receptor 36 with respect to a Cartesian coordinate system 40 centered on the receptor 36, and the third dimension being plotted as an amplitude or signal strength of the altered field plotted with respect to the two spatial dimensions. The processor may also in addition to or in lieu of the three dimensional representation present this information in one or two dimension(s). receptor To accurately identify the altered signal and discriminate the altered signal from noise, the signal 26 from the receptor(s) 22 may be passed through a frequency discriminator 42 and signal conditioner 44, which can be supplemented with or replaced with time gating techniques. Furthermore, digital signal processing techniques can also be used to achieve a better signal-to-noise ratio.

Returning to FIG. 1, it can be seen that in one embodiment of the claimed subject matter configured to be secured relative to the patient during the operation of the device; the system 10 may include a surgical tool guide 46 mounted to the carrier 32 for movement therewith so as to be accurately aligned with the geometric feature 12 when the system 10 has achieved a successful targeting. In this regard, the guide 46 preferably has a known location and orientation with respect to the emitter(s) 20 and receptor(s) 22 and the receptor 36 so that the guide 46 can be accurately aligned based on the targeting provided by the system 10. For example, the guide 46 may have a central tool guide axis 47 that will be aligned with a central axis 48 of the bore 12 when the bore 12 is accurately targeted by the system 10. Further, the emitter(s) 20 can be part of a removable insert in the tool guide 46, or can be located in the tool guide 46 itself, or both. Any suitable surgical tool guide 46 may be used, such as, for example, a guide for a surgical drill that can produce the appropriate holes in the femur 18 for allowing an anchor or fastener to be passed through the femur 18 and received in the one of the bores 12.

In another embodiment, the system 10 may also include a controller 50 to position the carrier 32 relative to the medical implant 14 in response to the representation of the altered field from the processor 24. In this regard, any suitable mechanical, electromechanical, and/or hydromechanical devices, many of which are known, can be used to translate and rotate the carrier 32 along and about any of the axes of the coordinate system 34. Additionally, depending upon the particular type of emitter(s) 20 and/or receptor(s) 22, the controller can adjust or vary the field from the emitter(s) 20, such as by varying the intensity or strength of the emitter field and/or the frequency of the emitter field, and adjust or vary the receptor parameters, such as adjusting the frequency of a tuned receptor 22.

In one embodiment of the claimed subject matter configured to be secured relative to the patient during the operation of the device; the controller 50 is manually controlled to position the carrier 32 in response to the representation of the altered field. In this embodiment, the system 10 further includes a user interface 52, preferably in the form of a visual display 54 (such as an LCD monitor) or an audio signal generator 56 (such as a speaker) or both, to transmit the representation of the altered field to a user/surgeon and, optionally, other information, such as warning signals, to the user. The user interface 52 also includes a user input 58 that allows the user to input positioning commands to the controller 50 to manually control the position of the carrier 32. Any suitable user input 58 can be used, many of which are known, including, for example, keyboards and/or joysticks.

As a separate embodiment, or in connection with the manual control discussed above in an embodiment of the claimed subject matter configured to be secured relative to the patient during the operation of the device; the controller 50 can also be configured to automatically position the carrier 32 in response to the representation of the altered field. In this regard, the controller 50 is programmed with a suitable targeting algorithm that “hunts” for the location and orientation of the geometric feature by either altering a frequency of the field from the emitter(s) 20 or altering the location of the emitter(s) 20 and receptor(s) 22 by translating and/or rotating the carrier 32 with respect to the implant 14, or by both altering the frequency and locations. In “hunting” for the location and orientation of the geometric feature 12, the controller 50 is attempting to generate a representation of the altered field that is similar to or matches an anticipated representation of the altered field based on the particular geometric feature, and type of implant. In this regard, the anticipated representation can either be a maximization of the strength of the signal in a predefined zone of the representation, or an anticipated shape of the representation, or both. For example, as one option, the controller 50 is responsive to relative changes in the intensity (represented by the amplitude) of the altered field with respect to the coordinate system 40. More specifically, the controller can be configured to translate and/or rotate the carrier 32 along and about any or all of the axes of the coordinate system 34 so as to center and/or align the receptor 36 and coordinate system 40 with either a zone of maximum intensity of the altered field or a zone of minimum intensity of the altered field, depending on the anticipated representation of the altered field. In one embodiment, the controller 50 may include a comparator 60 to compare at least a portion of the representation of the altered field to a shape of the known geometric feature 12 (or to an anticipated shape of the known geometric feature 12 based on how the known geometric feature 12 should alter the field) and the controller 50 is responsive to the comparator 60 to position the carrier 32 relative to the implant 14 based on the comparison. In one embodiment of the claimed subject matter, the comparator has stored within it geometrical information about the medical implant being used and allows the processor to identify any feature on the implant that would help with accurate location of the orientation of the medical implant. This stored information may be in the form of scanned data cloud, 3-D or 2-D images or any other form of data conveying geometrical information of standard medical implants.

In one embodiment of the “hunting” algorithm, in operation, the carrier 32 is positioned such that the center of the receptor 36 is approximately where the geometric feature 12 is expected to be based on the location of the patient 16 relative to the coordinate system 34. In this regard, for certain implants 14 it may be possible to determine the expected position of the geometric feature 12 based on an exposed or accessible portion of the implant 14. For example, the proximal end of an intramedullary nail is often exposed and/or accessible to a surgeon and can provide a reasonably accurate estimate of the location of the distal end and the predrilled bore adjacent thereto, particularly the location along the longitudinal axis of the nail because the distal end tends to wander laterally, with compression typically being an insignificant part of the deformation of the nail during implantation. If a portion of the implant 14 is not exposed or accessible for accurate location, a best estimate of the location of the geometric feature 12 can be made based on the location/position of the patient 16 with respect to the frame 30 and coordinate system 34. After the receptor 36 is initially positioned, the receptor(s) 22 detect the altered field and generate the signal 26 which is then passed to the processor 24 which generates the representation of the altered field, which may be in the form of the 3-D graph previously discussed. If the representation of the altered field doesn't appear to indicate the presence of the feature 12, the system 10 will attempt to locate the feature by translating the carrier 32 in the X, Y plane until an indication of the geometric feature 12, appears in the representation of the altered field. In this regard, it should be noted, that the carrier 32 can be translated along either the X axis or the Y axis or both. Alternately, depending on the particular type of emitter(s) 20 and receptor(s) 22, the system may simply alter the frequency of the field from the emitter(s) 20 until an indication of the geometric feature 12 appears in the representation of the altered field. Once the geometric feature 12 appears in the representation of the altered field, the system 10 then translates the carrier 32 to center the receptor coordinate system 40 with respect to the geometric feature 12 as determined by the representation of the altered field. The comparator 60 then compares the shape of the geometric feature 12 as shown in the representation of the altered field to an anticipated shape of the geometric feature 12 for the altered field. If the shapes do not match within a pre-programmed error or tolerance range, the controller 50 alters the position of the emitter(s) 20 and receptor(s) 22 by rotating about one or more of the axes of the coordinate system 34, either sequentially or in combination, until the representation of the altered field shows a suitable match for the anticipated shape of the geometric feature 12. For example, for a predrilled bore, the anticipated shape may be a circular cross section which can be seen in the plotted amplitudes of the altered signal if the receptor 36 is properly aligned with the bore, but which may appear as an ellipsoid if the receptor 36 has not yet been properly aligned with the bore. If the shape is an ellipsoid, the controller 50 will rotate the carrier about one of the X or Y axis, compare the shapes, and then rotate about the other of the X or Y axis, as appropriate, until the comparison of the shapes indicates a suitable match. Alternatively, the controller can manipulate the carrier 32 until the strength of the signal in a predefined zone of the representation of the altered field representing is maximized, as dictated by the anticipated representation of the altered field. One method to quantify the strength of the signal in the predefined zone is to measure the volume under the amplitude plot of the signal and to maximize that volume. While the foregoing has been described as an algorithm programmed into the controller 50, it should be understood that a surgeon/user could manually implement the algorithm via the user interface 52 if desired.

In an embodiment of the claimed subject matter configured to be secured relative to the patient during the operation of the device; once the geometric feature 12 has been accurately targeted by aligning the receptor 36 with the feature 12, such as by aligning the coordinate system 40 with the central axis 48 of a predrilled bore 12 in the distal end of an intramedullary nail 14, the surgical tool guide 46 can also be aligned, which will happen automatically if the tool guide 46 is mounted on the carrier 32 to be centered on the coordinate system 40. If an insert carrying the emitter(s) 20 has been mounted in the tool guide 46, the insert is replaced with a surgical tool, such as a surgical drill. Alternatively, if the emitter(s) 20 are retained in the tool guide 46 or the carrier 32, the emitter(s) 20 and receptor(s) 22 can continuously update the system 10 so as to insure accurate alignment of the surgical tool while the tool is utilized on the patient. In this regard, visual or audio warnings may be provided to the surgeon/user to indicate misalignment of the surgical tool together with an opportunity to correct the alignment.

Embodiments of the claimed subject matter as seen in FIG. 3, FIG. 4, FIG. 5 and FIG. 6 are configured such that the system need not be secured during it's operation relative to the patient.

With regards to FIG. 3 and FIG. 6, the carrier houses the emitter(s), detector(s), tool guide, at least one processor, at least one comparator and a user interface which may include a visual presentation of the altered signal and an audio signal generator.

Other previously described elements of the claimed subject matter configured to be secured relative to the patient during the operation of the device; may be incorporated in to the embodiments shown in FIG. 3, FIG. 4, FIG. 5 and FIG. 6. Frequency discriminator, signal conditioning and time gating techniques may be used as described supra.

The carrier as described in FIG. 3 and FIG. 6 may be configured to serve as an independent unit and may further include an on board power source and other elements described in the embodiment of the claimed subject matter configured to be secured relative to the patient during the operation of the device;

With regards to FIG. 4 and FIG. 5, the carrier is configured to house some elements of the system but not all the elements of the system.

In the embodiments described in FIG. 3, FIG. 4, FIG. 5 and FIG. 6, the claimed subject matter is configured to be hand held and operated by the user in the vicinity of the geometric feature to be located. The system would provide the user with at least one of a visual or audio representation of the altered geometric field. In one embodiment, the system of the claimed subject matter may further include a user input element which would allow the user among other options, an option to select from various representations of the altered geometric field. In the hand held embodiments of the claimed subject matter, the user performs various translations and rotations of the system in order to align the system appropriately based on the representation of the altered field.

In the aforementioned hand held embodiments of the claimed subject matter, the carrier is configured for ease of handling during operation of the system.

In one embodiment, at least one element of the claimed subject matter may be disposable. In another embodiment of the claimed subject matter, at least one element may be configured to be reusable. Further, elements of the system in the claimed subject matter may be configured to undergo sterilization between applications.

While any suitable emitter(s) 20 and detector(s) 22 can be utilized, in one form, the emitter(s) 20 is provided in the form of either a single constant DC field emitter or an array of DC field emitters (permanent magnets) that emit a magnetic field and the receptor is provided in either the form of a gaussmeter or an array of gaussmeters, or in the form of a magnetometer or an array of magnetometers. In another form, the emitter(s) 20 can be provided in the form of a pulse DC field emitter or an array of pulse DC field emitters (permanent magnets), with the receptor(s) 22 being provided either in the form of a gaussmeter or an array of gaussmeters or as a magnetometer or an array of magnetometers. In this regard, any suitable gaussmeter can be utilized, such as, for example, a Hall effect, Magnetoresistive, Magneticdiode, or Magnetotransistor. Similarly, any suitable magnetometer can be used, such as, for example, induction coil, air core loop antenna, rod antenna, fluxgate magnetometer. As yet another option, the emitter(s) 20 can be provided in the form of an AC magnetic field emitter (multi-frequency and pulsed eddy current techniques) or as an array of AC magnetic field emitters and the receptor(s) 22 can be provided in the form of a tuned AC magnetic field detector or an array of tuned AC magnetic field detectors or in the form of a capacitive detector or an array of capacitive detectors. As yet another option, the emitter(s) 20 can be provided in the form of an AC electric field emitter or an array of AC electric field emitters and the receptor(s) 22 can be provided in the form a tuned AC electric field detector or an array of tuned AC field detectors. As yet another option, the emitter(s) 20 can be provided in the form of an acoustic emitter or any array of acoustic emitters and the receptor(s) 22 can be provided in the form of an acoustic detector or an array of acoustic detectors. As a further option, the emitter could be provided in the form of an optical emitter or an array of optic emitters and the receptor could be provided in the form of an optical detector or an array of optical detectors. In this regard, it should be noted that as used herein, the word optical is considered to be a superset of the ultraviolet, visible, and infrared regimes of the electromagnetic spectrum of wavelength from 0.01 micrometers to 50 millimeters. In one embodiment of the claimed subject matter, the emitter(s) and receptor(s) may be located in a geometric pattern in a manner which optimizes the emission of the signals and the capturing of the altered signal.

While the system 10 has been described herein in connection with location and targeting of the predrilled bores in an intramedullary nail implanted in a patient, it should be understood that the claimed subject matter may find use with respect to other types of geometric features and/or other types of medical implants. Furthermore, it should be understood that the claimed subject matter may find use outside of the medical field for locating and targeting geometric features that are hidden or otherwise not easily located and targeted using standard visual techniques.

It will be appreciated that the system 10 allows for the targeting of a known geometric feature in a medical implant with emitter(s) 20 and receptor(s) 22 that are external to the implant. This also allows for the emitted field to be generated external to the implant. Furthermore the system 10 allows for the emitter(s) 20 and receptor(s) 22 to be located external to the patient so as not to require an invasive procedure for locating the emitter(s) 20 and/or receptor(s) 22 within the patient. In this regard, placing the emitter(s) 20 and/or receptor(s) 22 in an existing accessible cavity of the patient, such as in the mouth of a patient, is not considered to be an invasive procedure as the term is used herein.

1. A system to target a geometric feature in a medical implant located in a patient, the system comprising:
at least one emitter external to the implant to emit at least one of an acoustic field, a magnetic field, an electric field and an electromagnetic field that will be altered by the geometric feature;
at least one receptor external to the implant to generate a signal responsive to the altered field; and
a processor to receive the signal and generate a representation of the altered field.

2. The system of claim 1 further comprising a frame and a carrier mounted to the frame for translation and rotation along and about two axes relative to the medical implant, the at least one emitter and the at least one receptor mounted on the carrier for movement therewith along and about the two axes.

3. The system of claim 2 wherein the carrier is further mounted to the frame for translation and rotation along and about another axis relative to the medical implant.

4. The system of claim 2 wherein the at least one emitter and the at least one receptor are located on the carrier so that the medical implant can be positioned between the at least one receptor and the at least one emitter with the medical implant located in a patient.

5. The system of claim 2 wherein the at least one emitter and the at least one receptor are located on the carrier so that the medical implant can be positioned to one side of the at least one receptor and the at least one emitter with the medical implant located in a patient.

6. The system of claim 1 further comprising a comparator to compare at least a portion of the representation of the altered field to a shape of the known geometric feature.

7. The system of claim 2 further comprising a controller to position the carrier relative to the medical implant in response to the representation of the altered field.

8. The system of claim 7 wherein the controller comprises a comparator to compare at least a portion of the representation of the altered field to a shape of the known geometric feature, and the controller is responsive to the comparator to position the carrier relative to the implant.

9. The system of claim 7 wherein the controller is responsive to a representation of relative changes in an intensity of the altered field with respect to a reference coordinate system.

10. The system of claim 1 further comprising a surgical tool guide to be targeted at the geometric feature in response to the representation of the altered field.

11. The system of claim 2 further comprising a surgical tool guide mounted on the carrier to be targeted at the geometric feature in response to the representation of the altered field.

12. The system of claim 1 further comprising a user interface to transmit the representation of the altered field to a user.

13. The system of claim 12 wherein the user interface comprises at least one of a visual display and an audio signal.

14. The system of claim 1 wherein the at least one emitter comprises a constant DC field emitter.

15. The system of claim 1 wherein the at least one emitter comprises a pulsed DC field emitter.

16. The system of claim 1 wherein the at least one emitter comprises an AC magnetic field emitter.

17. The system of claim 1 wherein the at least one emitter comprises an AC electric field emitter.

18. The system of claim 1 wherein the at least one emitter comprises an acoustic emitter.

19. The system of claim 1 wherein the at least one emitter comprises an optical emitter.

20. A method to target a geometric feature in a medical implant located in a patient, the method comprising:
emitting, at a location external to the implant, at least one of an acoustic field, a magnetic field, an electric field and an electromagnetic field that will be altered by the geometric feature;
detecting the altered field at a location external to the implant; and
generating a representation of the altered field in response to detection of the altered field detection.

21. The method of claim 20 further including altering the locations of the emitting and detecting relative to the medical implant in response to the representation of the altered field.

22. The method of claim 21 wherein altering the locations comprises translating the locations along at least one axis relative to the medical implant.

23. The method of claim 21 wherein altering the location comprises rotating the locations about at least one axis relative to the medical implant.

24. The method of claim 20 wherein emitting comprises varying the at least one of an acoustic field, a magnetic field, an electric field and an electromagnetic field.

25. The method of claim 24 wherein varying comprises varying a frequency of the at least one of an acoustic field, a magnetic field, an electric field and an electromagnetic field.

26. The method of claim 24 wherein varying comprises pulsing the at least one of an acoustic field, a magnetic field, an electric field and an electromagnetic field.

27. The method of claim 20 further comprising comparing at least a portion of the representation of the altered field to a shape of the known geometric feature.

28. The method of claim 27 further comprising altering the locations of emitting and detecting relative to the medical implant in response to comparing.

29. The method of claim 28 wherein altering comprises translating the locations along at least one axis relative to the medical implant.

30. The method of claim 28 wherein altering comprises rotating the locations about at least one axis relative to the medical implant.

31. The method of claim 20 further comprising comparing relative changes in an intensity of the representation of the altered field with respect to a reference coordinate system.

32. The method of claim 31 further comprising altering the locations of the emitting and detecting relative to the medical implant in response to comparing.

33. The method of claim 32 wherein altering comprises translating the locations along at least one axis relative to the medical implant.

34. The method of claim 32 wherein altering comprises rotating the locations about at least one axis relative to the medical implant.

35. The method of claim 20 further comprising targeting a surgical tool guide relative to the geometric feature in response to generating.

36. The method of claim 20 further comprising transmitting the representation of the altered field to a user.

37. The method of claim 36 wherein transmitting at least a portion of the representation of the field to at least one of a visual display and an audio signal.

38. A system to target a geometric feature in a medical implant located in a patient, the system comprising:
at least one emitter to emit at least one of an acoustic field, a magnetic field, an electric field and an electromagnetic field that will be altered by the geometric feature;
at least one receptor to generate a signal responsive to the altered field;
a carrier which houses the at least one emitter and the at least one receptor external to the implant.

39. The system of claim 38, further comprising a processor housed in the carrier to receive the signal and generate a representation of the altered field.

40. The system of claim 38, further comprising a processor located external to the carrier to receive the signal and generate a representation of the altered field.

41. The system of claim 39, wherein the representation of the altered field comprises information of at least one of the intensity, shape, frequency and phase of the altered field.

42. The system of claim 40, wherein the representation of the altered field comprises information of at least one of the intensity, shape, frequency and phase of the altered field.

42. The system of claim 38, further comprising a comparator located in the carrier, to compare at least a portion of the representation of the altered field to a shape of the known geometric feature.

43. The system of claim 38, further comprising a comparator located external to the carrier, to compare at least a portion of the representation of the altered field to a shape of the known geometric feature.

44. The system of claim 38, further comprising a tool guide mounted on the carrier to be targeted relative to the geometric feature in response to the representation of the altered field.

45. The system of claim 38, further comprising a user interface housed in the carrier to transmit the representation of the altered field to a user.

46. The system of claim 38, further comprising a user interface external to the carrier to transmit the representation of the altered field to a user.

47. The system of claim 45, wherein the user interface comprises at least one of a visual display and an audio signal.

48. The system of claim 46, wherein the user interface comprises at least one of a visual display and an audio signal.

49. The system of claim 38, wherein at least a portion of the system is disposable.
